(19) United States
(12) Patent Application Publication
(10) Pub. No.: US 2024/0081700 A1
(43) Pub. Date: Mar. 14, 2024

(54) METHOD AND SYSTEM FOR PROVIDING CONTINUOUS CALIBRATION OF IMPLANTABLE ANALYTE SENSORS

(71) Applicant: ABBOTT DIABETES CARE INC., Alameda, CA (US)

(72) Inventor: Benjamin Jay Feldman, Berkeley, CA (US)

(73) Assignee: ABBOTT DIABETES CARE INC., Alameda, CA (US)

(21) Appl. No.: 18/512,733

(22) Filed: Nov. 17, 2023

Related U.S. Application Data
(63) Continuation of application No. 16/181,075, filed on Nov. 5, 2018, now Pat. No. 11,872,039.
Application No. 16/181,075 is a continuation of application No. 14/850,957, filed on Sep. 11, 2015, now Pat. No. 10,117,614.
Application No. 14/850,957 is a continuation of application No. 13/959,302, filed on Aug. 5, 2013, now abandoned.
Application No. 13/959,302 is a continuation of application No. 13/022,620, filed on Feb. 7, 2011, now Pat. No. 8,506,482.
Application No. 13/022,620 is a continuation of application No. 11/365,340, filed on Feb. 28, 2006, now Pat. No. 7,885,698.

(51) Int. Cl. A61B 5/1495; A61B 5/00; A61B 5/145; A61M 5/172; G16H 50/20; H04L 67/12

(52) U.S. Cl. CPC A61B 5/1495; A61B 5/0017; A61B 5/0024; A61B 5/0031; A61B 5/14503; A61B 5/6846; A61B 5/7246; A61M 5/1723; G16H 50/20; H04L 67/12; A61B 5/14532; A61B 2560/0223

(57) ABSTRACT

Method and system for providing continuous calibration of analyte sensors includes calibrating a first sensor, receiving data associated with detected analyte levels from the first sensor, and calibrating a second sensor based on a predetermined scaling factor and data associated with detected analyte levels from the first sensor, is disclosed.

### RELATED APPLICATIONS

The present application is a continuation of U.S. patent application Ser. No. 16/181,075 filed Nov. 5, 2018, which is a continuation of U.S. patent application Ser. No. 14/850,957 filed Sep. 11, 2015, now U.S. Pat. No. 10,117,614, which is a continuation of U.S. patent application Ser. No. 13/959,302 filed Aug. 5, 2013, which is a continuation of U.S. patent application Ser. No. 13/022,620 filed Feb. 7, 2011, now U.S. Pat. No. 8,506,482, which is a continuation of U.S. patent application Ser. No. 11/365,340 filed Feb. 28, 2006, now U.S. Pat. No. 7,885,698, entitled “Method and System for Providing Continuous Calibration of Implantable Analyte Sensors”, the disclosure of which is incorporated herein by reference for all purposes.

### BACKGROUND

Continuous monitoring of analytes of a patient generally uses an analyte sensor that that is at least partially implanted in the patient so as to be in fluid contact with the patient's analytes such as interstitial fluid or blood. The analyte sensor typically is replaced after a predetermined time period such as three, five or seven day period, when a new sensor is implanted in the patient to replace the old sensor. During the sensor replacement process, a gap or interruption in the analyte monitoring occurs. For example, during the time period in which the patient removes the implanted analyte sensor to replace with a new analyte sensor, the patient is unable to monitor or determine the analyte values such as glucose levels. In this manner, with continuous glucose monitoring systems presently available which use short term analyte sensors, there is always a gap in service during which data associated with the measurement of the patient's analyte levels cannot be obtained.

In addition, calibration of each implanted analyte sensor, which is necessary before data from the analyte sensor can be obtained, is laborious, time consuming, and error prone. Factory calibration is not a practical approach due to substantial sensor to sensor variability of signal strength introduced during the manufacturing process, and also, due to additional variability imposed by the sensors' response to the in-vivo environment which varies from patient to patient.

Thus, typically it is necessary to perform in-vivo calibration, in which the analyte sensor is calibrated, post implantation, by comparison with a reference blood glucose value. Generally these reference blood glucose values include capillary blood glucose values obtained by finger or arm stick using a conventional blood glucose meter. To perform the calibration using the reference blood glucose values, a substantial number of capillary values such as, for example, one to four capillary measurements daily, are necessary to ensure the continued calibration (and thus, accurate) values determined by the analyte sensors.

Moreover, calibrations may sometimes be inaccurate due to transient sensitivity changes which generally occur early in the lifetime of an implanted sensor, and sometimes referred to as early sensitivity attenuation, or ESA. If a calibration is assigned to an analyte sensor undergoing a transient change in sensitivity, inaccurate sensor readings or measurements will result at a later point in time, when the sensitivity reverts to its “true” value.

Further, the typical calibration process is performed for each newly implanted glucose sensor. More specifically, with the placement of each glucose sensor, a new set of blood capillary reference values are obtained, and which is the sole basis (or reference) for calibration of that particular sensor during the usage life of the sensor, for example, during a three, five or a seven day period.

In view of the foregoing, it would be desirable to have an approach to provide methods and system for continuous analyte monitoring where no gap in service can be achieved. In addition, it would be desirable to have methods and a system to verify the stability of a newly implanted sensor, before obtaining user-accessible analyte data from the sensor. Furthermore, it would be desirable to have methods and system for continuous analyte monitoring for continuous calibration of analyte sensors and which minimizes the number of necessary fingerstick (or armstick) calibrations of the analyte sensors using glucose meters, and also, to provide alternate reference.

### SUMMARY OF THE INVENTION

In view of the foregoing, in accordance with the various embodiments of the present invention, there is provided a method and system in which short term sensors may be calibrated based on the data associated with prior short term sensors by providing an overlap in the sensor placement during the sensor replacement process such that fewer, or in the limit, no additional capillary blood glucose values are needed for calibration of subsequent sensors, and further, analyte levels are continuously monitored without any interruption, for example, during the periodic sensor replacements in the continuous analyte monitoring system.

These and other objects, features and advantages of the present invention will become more fully apparent from the following detailed description of the embodiments, the appended claims and the accompanying drawings.

### BRIEF DESCRIPTION OF THE DRAWINGS

FIG. 1 is a block diagram illustrating a continuous analyte monitoring system for practicing one embodiment of the present invention;

FIG. 2 is a flowchart illustrating the continuous calibration of analyte sensors in the continuous analyte monitoring system in accordance with one embodiment of the present invention;

FIG. 3 is a flowchart illustrating correlation and calibration steps 230, 240 of the continuous calibration of analyte sensors in the continuous analyte monitoring system shown in FIG. 2 in accordance with one embodiment of the present invention;

FIG. 4 is a chart illustrating the timing of the continuous calibration of analyte sensors in the continuous analyte monitoring system in accordance with one embodiment of the present invention;

FIG. 5 is a chart illustrating the measured analyte values of a first calibrated analyte sensor in the continuous analyte monitoring system in accordance with one embodiment of the present invention;

FIG. 6 is a chart illustrating the measured analyte values of a second analyte sensor which is implanted while the calibrated first analyte sensor is implanted in the continuous analyte monitoring system in accordance with one embodiment of the present invention;

FIG. 7 is a chart illustrating the measured analyte values of the second analyte sensor which is calibrated and correlated with the measured values from the calibrated analyte sensor in accordance with one embodiment of the present invention; and

FIG. 8 is a chart illustrating measured analyte values after the removal of the first analyte sensor, and from the calibrated second analyte sensor in accordance with one embodiment of the present invention.

### DETAILED DESCRIPTION

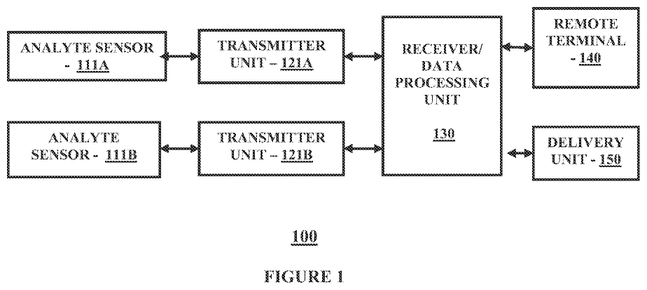

FIG. 1 is a block diagram illustrating a continuous analyte monitoring system for practicing one embodiment of the present invention. Referring to FIG. 1, a continuous analyte monitoring system 100 includes analyte sensor 111A operatively coupled to a transmitter unit 121A, and analyte sensor 111B operatively coupled to transmitter unit 121B. Further shown is a receiver/data receiving unit 130 which is operatively coupled to transmitter unit 121A and transmitter unit 121B. The receiver/data processing unit 130 in one embodiment is configured to communicate with a remote terminal 140 and a delivery unit 150. The remote terminal 140 in one embodiment may include for example, a desktop computer terminal, a data communication enabled kiosk, a laptop computer, a handheld computing device such as a personal digital assistant (PDAs), or a data communication enabled mobile telephone. Moreover, the delivery unit 150 may include in one embodiment, but not limited to, an external infusion device such as an external insulin infusion pump, an implantable pump, a pen-type insulin injector device, a patch pump, an inhalable infusion device for nasal insulin delivery, or any other type of suitable delivery system.

Referring to FIG. 1, the receiver/data receiving unit 130 is configured to receive analyte related data from transmitter unit 121A and transmitter unit 121B over a wireless data communication link such as, but not limited to, radio frequency (RF) communication link, Bluetooth® communication link, infrared communication link, or any other type of suitable wireless communication connection between two or more electronic devices which may further be uni-directional (e.g., from transmitter units 121A, 121B to receiver/data processing unit 130), or alternatively, bi-directional between the two or more devices. Alternatively, the data communication link connecting the transmitter units 121A and 121B to the receiver/data processing unit 130 may include wired cable connection such as, for example, but not limited to RS232 connection, USB connection, or serial cable connection.

In an alternate embodiment, each of the transmitter units 121A and 121B may be individually coupled to a corresponding receiver section (for example, separate receiver/data processing sections of the receiver/data processing unit 130) such that each transmitter unit 121A and 121B are uniquely operatively coupled to the respective receiver/data processing units. In addition, each receiver/data processing unit may be configured to communicate with each other such that data from the transmitter units 121A and 121B may be interchangeably communicated. Furthermore, while FIG. 1 illustrates a single receiver/data processing unit 130, within the scope of the present invention, multiple discrete receiver/data processing units may be provided, each uniquely configured to communicate with a corresponding one of the transmitter units 121A, 121B.

Furthermore, in yet another embodiment of the present invention, the transmitter unit 121A and transmitter unit 121B may be physically coupled in a single housing so as to provide a single transmitter section for the patient, which is configured to support multiple transmitter units 121A, 121B. Moreover, while two transmitter units 121A, 121B are shown in FIG. 1, within the scope of the present invention, the continuous analyte monitoring system 100 may be configured to support additional and/or multiple transmitter units, multiple remote terminals, and receiver/data processing units.

Moreover, referring to FIG. 1, the analyte sensors 111A and 111B may include, but are not limited to short term subcutaneous analyte sensors or transdermal analyte sensors, for example, which are configured to detect analyte levels of a patient over a predetermined time period, and after which, a replacement of the sensors is necessary. Moreover, in one embodiment, the transmitter units 121A, 121B are configured to receive analyte related data from the corresponding analyte sensors 111A, 111B, respectively, and to transmit data to the receiver/data processing unit 130 for further processing.

The transmitter units 121A, 121B may, in one embodiment, be configured to transmit the analyte related data substantially in real time to the receiver/data processing unit 130 after receiving it from the corresponding analyte sensors 111A, 111B respectively. For example, the transmitter units 121A, 121B may be configured to transmit once per minute to the receiver/data processing unit 130 based on analyte levels detected by the corresponding analyte sensors 111A, 111B respectively. While once per minute data transmission is described herein, within the scope of the present invention, the transmitter units 121A, 121B may be configured to transmit analyte related data more frequently (such as, for example, once every 30 seconds), or less frequently (for example, once every 3 minutes).

Additional analytes that may be monitored, determined or detected by analyte sensors 111A, 111B include, for example, acetyl choline, amylase, bilirubin, cholesterol, chorionic gonadotropin, creatine kinase (e.g., CK-MB), creatine, DNA, fructosamine, glucose, glutamine, growth hormones, hormones, ketones, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be determined.

Moreover, within the scope of the present invention, transmitter units 121A, 121B may be configured to directly communicate with one or more of the remote terminal 140 or the delivery unit 150, and in addition, the receiver/data processing unit 130 may be integrated with one or more of the remote terminal 140 or the delivery unit 150. Furthermore, within the scope of the present invention, additional devices may be provided for communication in the continuous analyte monitoring system 100 including additional receiver/data processing unit, remote terminals (such as a physician's terminal) and/or a bedside terminal in a hospital environment, for example.

In accordance with the various embodiments of the present invention, the analyte sensors 111A, 111B may be inserted through the skin of the patient using insertion devices having a predefined or configured insertion mechanism (spring loaded devices, for example) which facilitates the placement and positioning of the analyte sensors through the patient's skin, and so as to be in fluid contact with the patient's analytes. Alternatively, the sensors 111A, 111B may be manually deployed using an insertion guide or needle.

As described in further detail below, the continuous calibration process in one embodiment includes deploying and calibrating a first sensor (e.g., analyte sensor 111A) at predetermined time intervals using fingerstick calibrations, for example, at 10 hours, 12 hours, 24 hours and 72 hours from the initial insertion of the first sensor 111A. Moreover, the first and subsequent analyte measurements may be obtained after the initial calibration at 10 hours when the analyte sensor has substantially reached a stability point. Thereafter, prior to the termination of the first sensor life (for example, at the 120^{th} hour for a 5 day sensor), a second analyte sensor (for example, sensor 111B) is inserted into the patient and during the period of overlap of the first and second analyte sensors 111A, 111B, the second analyte sensor 111B is correlated with the first analyte sensor 111A values and the second analyte sensor 111B is calibrated in reference to the first analyte sensor 111A values such that the second analyte sensor 111B and any additional subsequent analyte sensors do not require the multiple (or preferably, any) fingerstick calibrations as is the case for the first analyte sensor 111A.

In this manner, the short term analyte sensors are overlapped for a predetermined time period to allow the output of the first and second sensors to be correlated to detect potential transient sensitivity (e.g., ESA) in the second sensor. The detection of potential transient sensitivity in the second sensor can be achieved with substantial accuracy since the first sensor has had a substantial time period (e.g., several days of usage) to stabilize. Upon establishing an acceptable level of correlation, the calibration of the first sensor in one embodiment is assigned or transferred to the second sensor. More specifically, in one embodiment, the continuous data from a previously calibrated first sensor is used as a set of reference values to calibrate the second newly implanted sensor.

In this manner, in one embodiment of the present invention, a substantially accurate calibration may be assigned to the second sensor while using no additional capillary blood glucose values for calibration, and further, this approach of correlation and transfer calibration may be repeated for subsequent sensors in the continuous monitoring system 100 such that analyte levels are continuously monitored without any interruption, for example, during the periodic sensor replacements in the continuous analyte monitoring system 100.

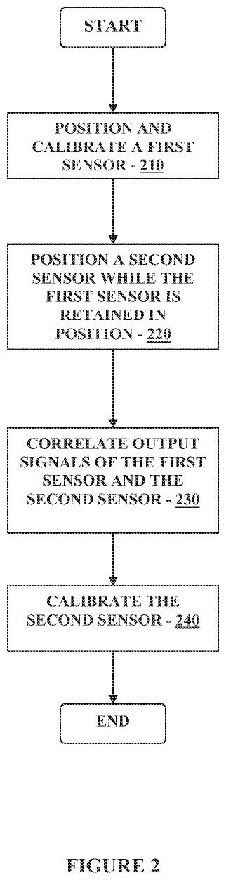

FIG. 2 is a flowchart illustrating the continuous calibration of analyte sensors in the continuous analyte monitoring system in accordance with one embodiment of the present invention. Referring to FIG. 2, at step 210 a first sensor 111A (FIG. 1) is deployed through the patient's skin so as to be in fluid contact with the patient's analyte and periodically calibrated at, for example, 10^{th} hour, 12^{th} hour, 24^{th} hour, and 72^{nd} hour. Data associated with the detected or monitored analyte level from the first sensor 111A may be obtained for further analysis such as insulin therapy and treatment on or after the 10^{th} hour calibration when the first sensor has substantially reached an acceptable stabilization level.

More specifically, the transmitter unit 121A (FIG. 1) is configured in one embodiment to continuously transmit the data received from the analyte sensor 111A to the receiver/data processing unit 130 (FIG. 1). The receiver/data processing unit 130 may be configured in one embodiment, to display the received, substantially real time values corresponding to the patient's monitored analyte levels graphically, audibly, and/or a combination of visual and audio output including graphs, trend arrows, and level indicators associated with different sound levels or ringtones based on the analyte levels.

Referring to FIG. 2, at step 220, a second analyte sensor 111B is positioned at a predetermined time prior to the scheduled removal of the first analyte sensor 111A. In one embodiment, the predetermined time overlap between the insertion of the second analyte sensor 111B and the removal of the first analyte sensor 111A from the patient may be a two to ten hour period. Alternatively, the time overlap may be longer or shorter depending upon the sensor configuration and the preceding time periods are provided as examples for illustrative purposes only. In one embodiment, the time overlap may be variable, such that first analyte sensor 111A is removed when second analyte sensor 111B is determined to have reached a point of stable operation. Thereafter, at step 230, the output data or signals from the first sensor 111A received from transmitter unit 121A is correlated with the output data or signals from the second sensor 111B received from the transmitter unit 121B. That is, the receiver/data processing unit 130 (FIG. 1) in one embodiment is configured to receive the simultaneous or substantially near simultaneous data transmission from a plurality of transmitter units in the continuous analyte monitoring system 100. More specifically, in one embodiment, the receiver/data processing unit 130 may be configured to correlate the data from the two sensors 111A, 111B so as to, for example, determine that the correlation of the two data sets are sufficiently robust to determine the stability and thus acceptability of the second sensor 111B.

Referring again to FIG. 2, after correlating the data of the two sensors 111A, 111B, at step 240, the second sensor is calibrated based on one or more scaling factors associated with the two sensors 111A, 111B, from which the sensitivity of the second sensor 111B may be determined. Thereafter, when the second sensor 111B is calibrated, the first sensor 111A may be removed from the patient, and the data or signals associated with the patient's analyte levels from the second sensor 111B may be used by the patient for further analysis and/or treatment.

In the manner described above, in one embodiment of the present invention, there is provided a system and method of continuously calibrating implanted analyte sensors that provide accurate detection of initial instabilities of the implanted sensors, reduce the number of required blood capillary tests for calibration, increase the calibration accuracy, and also, eliminate any gaps or interruptions in the continuous analyte data or record monitored by the continuous monitoring system 100.

Moreover, in a further embodiment, the receiver/data processing unit 130 may be configured to prompt the patient for confirmation and also, for the sensor calibration code when the receiver/data processing unit 130 detects data or signals received from the transmitter unit 121B coupled to the second sensor 111B.

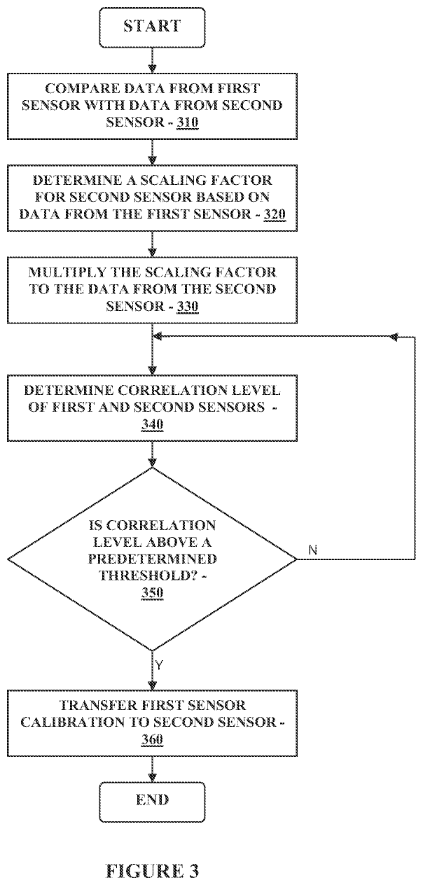

FIG. 3 is a flowchart illustrating correlation step 230 and calibration step 240 of the continuous calibration of analyte sensors in the continuous analyte monitoring system shown in FIG. 2 in accordance with one embodiment of the present invention. Referring to FIG. 3, at step 310, the receiver/data processing unit 130 (FIG. 1) is configured to compare the analyte associated data or signals from the transmitter unit 121A corresponding to analyte levels detected by the first sensor 111A with the analyte associated data or signals from the transmitter unit 121B corresponding to analyte levels detected by the second sensor 111B at each time period of the analyte monitoring.

Thereafter, at step 320, the receiver/data processing unit 130 is configured to determine a scaling factor for the second sensor 111B based on the data or signals from the first sensor 111A. More specifically, in one embodiment, the receiver/data processing unit 130 is configured to perform a predefined autocorrelation function to determine the scaling factor for the second sensor 111B. Alternatively, in another embodiment, the data from the second sensor 111B is multiplied by a range of predetermined initial scaling factors to determine an average error between the data from the first sensor 111A and the data from the second sensor 111B. Based on the calculated average error, the scaling factor is determined as the one of the predetermined initial scaling factors which yields the smallest possible calculated average error.

In a further embodiment, the scaling factor may be determined by calculating an average of the ratio of the two raw signals from the first sensor 111A and the second sensor 111B, or any other suitable manner in which to determine a suitable scaling factor.

Referring to FIG. 3, after the scaling factor is determined at step 320, the scaling factor is applied to the data from the second sensor 111B at step 330. More specifically, in one embodiment, the determined scaling factor at step 320 is multiplied to the data from the second sensor 111B at step 330. Thereafter, at step 340, a correlation level of the first and second sensors 111A, 111B respectively, is determined by the receiver/data processing unit 130 (FIG. 1). More specifically, at step 340, the level of correlation of data from the first sensor 111A and the second sensor 111B are determined as a function of a predetermined limit, where, in the case where the level of correlation is too small such that the minimum average error is too large, it is determined that the second sensor 111B is unstable.

In other words, referring back to FIG. 3, at step 340, the correlation level is determined and at step 350, it is determined whether the correlation level is above a predetermined threshold. As shown in the Figure, if it is determined at step 350 that the correlation level is not above the predetermined threshold level, then the receiver/data processing unit 130 returns the routine to step 340 to determine again the correlation level of the first sensor 111A and the second sensor 111B. If at step 350 it is determined that the correlation level is above the predetermined threshold level, then the receiver/data processing unit 130 determines that the second sensor 111B is relatively stable, and at step 360, the first sensor 111A calibration is transferred to the second sensor 111B.

In other words, once it is determined that the second sensor 111B is stable, then a sensitivity may be determined for the second sensor 111B based on the scaling factor determined at step 320 and the sensitivity of the first sensor 111A. This determination may be expressed as follows:

S₂=S₁*Σ(I₂/I₁) (1)

where S₂ represents the sensitivity of the second sensor 111B, S₁ represents the sensitivity of the first sensor 111A, and Σ(I₂/I₁) represents the scaling factor which correlates the data of the first sensor 111A and the second sensor 111B.

In this manner, once the second sensor 111B is calibrated, the accuracy of data from the second sensor 111B is substantially similar to the accuracy of the data from the first sensor 111A, where the calibration of the second sensor 111B was performed without any capillary blood glucose measurements by, for example, fingerstick testing using glucose meters. By way of an example, based on a first sensor sensitivity S₁ at 0.686 nA/mM, and with a scaling factor Σ(I₂/I₁) of 0.725, the sensitivity S₂ of the second sensor 111B is determined to be 0.497 nA/mM.

In the manner described above, in one embodiment of the present invention, there is provided a system and method of continuously calibrating implanted analyte sensors that provide accurate detection of initial instabilities of the implanted sensors, reduce the number of required blood capillary tests for calibration, increase the calibration accuracy, and also, eliminate any gaps or interruptions in the continuous analyte data or record monitored by the continuous monitoring system 100.

Moreover, in accordance with the present invention, using the data correlation during the time period when the sensors overlap in time, the calibration frequency may be reduced while increasing the calibration accuracy. Moreover, additional calibration information may also be obtained from the sensor calibration codes predetermined and assigned during sensor manufacturing, and which may be used to improve calibration accuracy without requiring additional or increased capillary blood glucose testing.

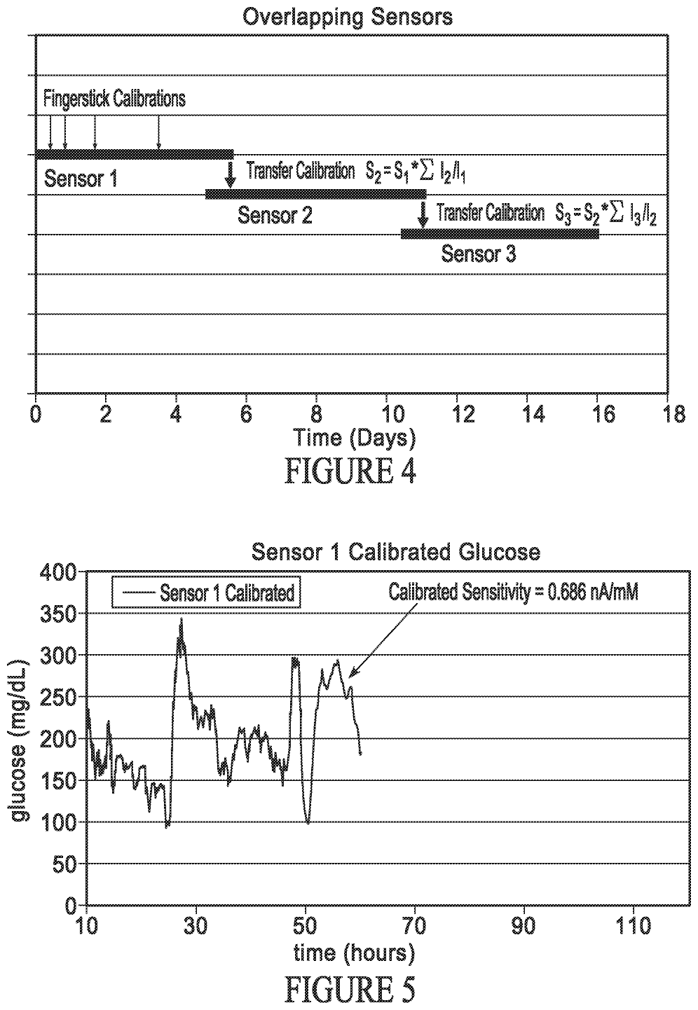

FIG. 4 is a chart illustrating the timing of the continuous calibration of analyte sensors in the continuous analyte monitoring system in accordance with one embodiment of the present invention. Referring to the Figure, each sensor is configured to be approximately a 5-day sensor, with only the first sensor (sensor 1) provided with four discrete fingerstick calibrations using capillary blood glucose measurements. It can be further seen that each sensor overlaps in time such that a predetermined time period overlaps after the insertion and positioning of a subsequent sensor, and before to the removal of the prior sensor. Moreover, calibration of sensor 2 and sensor 3 (and additional sensors thereafter) are performed based on the continuous calibration approach described above using the data correlation and transfer calibration as described.

FIG. 5 is a chart illustrating the measured analyte values of a first calibrated analyte sensor in the continuous analyte monitoring system in accordance with one embodiment of the present invention. Referring to FIG. 5, it can be seen that over the initial 60 or so hours of glucose level measurements, and based on the fingerstick calibration, the calibrated sensitivity S₁ of the first sensor may be determined (for example, at 0.686 nA/mM). Thereafter, as described above, the sensitivity of the second and subsequent sensors may be determined based on the first sensor sensitivity S₁ and the optimal scaling factor.

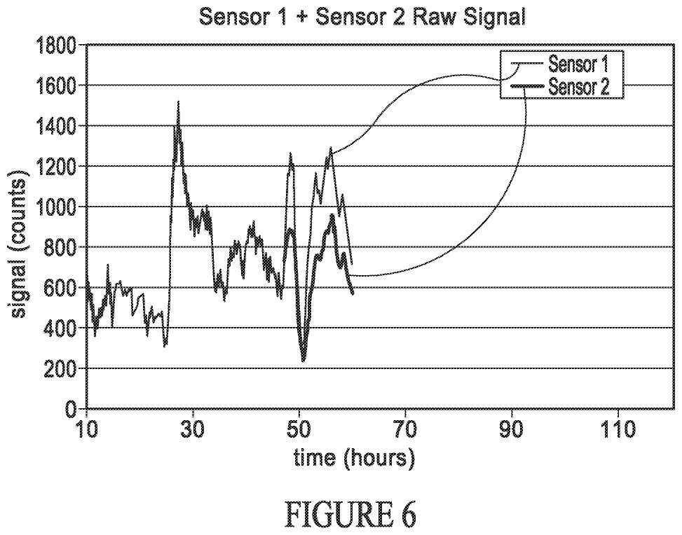

FIG. 6 is a chart illustrating the measured analyte values of a second analyte sensor which is implanted while the calibrated first analyte sensor is implanted in the continuous analyte monitoring system in accordance with one embodiment of the present invention. More specifically, FIG. 6 illustrates, in an overlay manner, the calibrated signals from the first sensor 111A and uncalibrated signals from the second sensor 111B received by the receiver/data processing unit 130 over the time period during which the first sensor 111A is retained in inserted position, and while the second sensor 111B is introduced in the patient. It should be noted that the signal count as shown on the Y-axis may be converted to a current signal level by a multiplication factor of 11.5 picoamps/count.

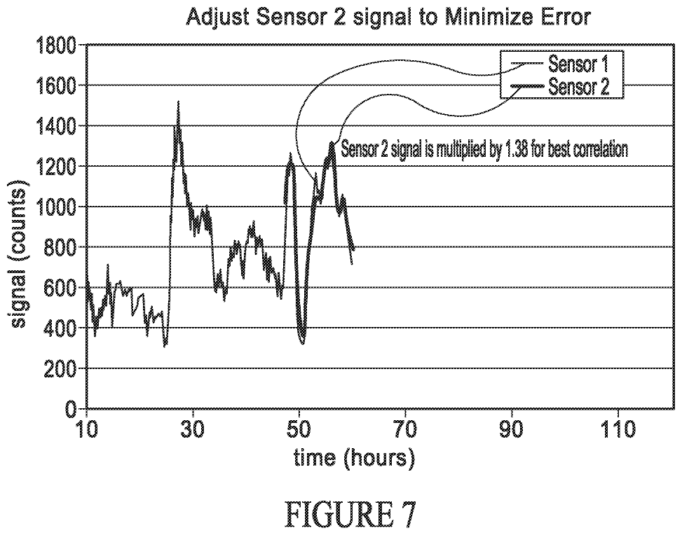

FIG. 7 is a chart illustrating the measured analyte values of the second analyte sensor which is calibrated and correlated with the measured values from the calibrated analyte sensor in accordance with one embodiment of the present invention. More specifically, as can be seen from FIG. 7, in the scaling factor and the correlation of the data from the second sensor 111B with the calibrated data from the first sensor 111A substantially aligns the two data sets over the overlap time period, effectively, providing calibration to the raw data from the second sensor 111B based on the calibrated data from the first sensor 111A.

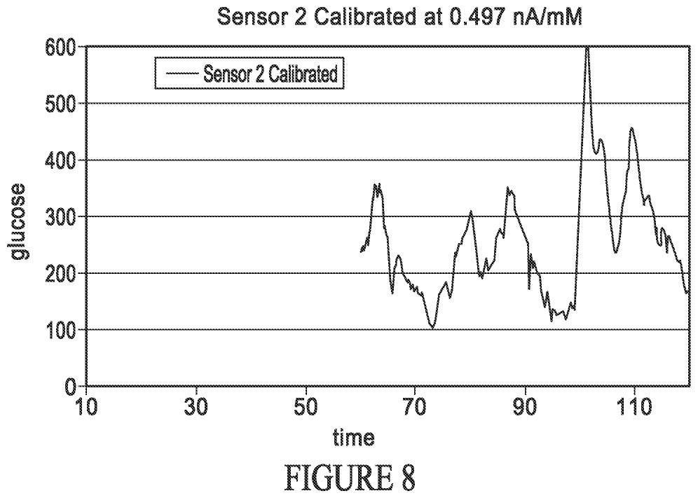

FIG. 8 is a chart illustrating measured analyte values after the removal of the first analyte sensor, and from the calibrated second analyte sensor in accordance with one embodiment of the present invention. In FIG. 8, it can be seen that the first sensor 111A is removed from the patient and thus the receiver/data processing unit 130 (FIG. 1) no longer receives data from the transmitter unit 121A coupled to the sensor 111A. On the other hand, the second sensor 111B is now calibrated and the data received from the second sensor 111B is received by the receiver/data processing unit 130. In this manner, it can be seen that there is no interruption in the measured analyte levels even during the transition state where the short term sensors are replaced.

Accordingly, a method of providing continuous calibration of analyte sensors in one embodiment of the present invention includes calibrating a first sensor, receiving data associated with detected analyte levels from the first sensor, and calibrating a second sensor with reference to one or more detected analyte levels from the first sensor.

The method in one embodiment may further include a step of calibrating a third sensor based on a second scaling factor and data associated with detected analyte levels from the second sensor. Moreover, the step of calibrating the second sensor may in one embodiment, start after a predetermined time period has passed where the first sensor has been in fluid contact with an analyte of a patient, where the predetermined time period may include at least approximately 90% or alternatively, 50% of the life of the first sensor.

In yet another embodiment, the method may further include the step of determining a sensitivity of the first sensor.

In another aspect, the method may also include the step of receiving data associated with detected analyte levels from the second sensor.

In accordance with still another embodiment, the step of calibrating the second sensor may include the steps of determining an analyte level, and comparing the determined analyte level with the data associated with the detected analyte level from the first sensor.

The step of calibrating the second sensor in yet another embodiment may include the steps of determining a scaling factor based on substantially simultaneous data from the first sensor and the second sensor, applying the scaling factor to the data from the second sensor, determining a correlation level of data from the first sensor and from the second sensor.

In another aspect, the step of determining the scaling factor may include the steps of comparing the substantially simultaneous data from the first sensor with the data from the second sensor, and determining the scaling factor based on a calculated scaling factor with the lowest level of average error between the data of the first sensor and the data of the second sensor.

The method in yet another embodiment may include the step of comparing the correlation level with a predetermined correlation threshold defining an acceptable stability level of the second sensor.

The first sensor and the second sensor may be analyte sensors.

This may further include the step of removing the first sensor while retaining the second sensor in fluid contact with the analyte of a patient.

In addition, the first sensor and the second sensor may be subcutaneously positioned under a skin of a patient, where at least a portion of the first sensor and at least a portion of the second sensor are in fluid contact with the patient's analyte.

A system for providing continuous analyte sensor calibration in accordance with another embodiment of the present invention includes a first sensor for subcutaneous placement in a patient, a second sensor for subcutaneous placement in the patient after calibration of the first sensor, where at least a portion of the first sensor and at least a portion of the second sensor are in fluid contact with the patient's analyte substantially simultaneously for a time period.

In one aspect, the time period may be predetermined and includes approximately 2 hours to 10 hours.

Alternatively, in another aspect, the time period may be variable, and where the variable time period may be determined to be when the analyte levels measured by the first and second sensors are within a correlation range, the correlation range being determined by a preset threshold value.

The second sensor may subcutaneously placed in the patient after a predetermined time period has passed where the first sensor has been in fluid contact with an analyte of a patient, and where the predetermined time period includes at least approximately 90% or 50% of the life of the first sensor.

In a further embodiment, the first sensor may be operatively coupled to a first transmitter unit, and the second sensor is operatively coupled to a second transmitter unit, the first and second transmitter units configured to receive data from the corresponding first and second sensors, respectively, for transmission over a communication link.

The first transmitter unit and the second transmitter unit may be coupled to a single transmitter housing.

The communication link may include one or more of an RF communication link, a Bluetooth® communication link, an infrared communication link, or a cable communication link.

The system in another embodiment may include a receiver unit configured to substantially simultaneously receive data from the first transmitter unit and the second transmitter unit.

The receiver unit may include a first receiver section operatively coupled to the first transmitter unit, and a second receiver section operatively coupled to the second transmitter unit, the second receiver section further operatively coupled to the first receiver section for data communication.

The receiver unit may also include an infusion device.

The receiver unit may be configured to calibrate the second sensor based on analyte levels measured by the first sensor.

The receiver unit may be configured to receive data from one or more of the first sensor or the second sensor at predetermined time intervals such that there is no interruption in the received data after the first sensor is removed from the patient.

A method in another embodiment of the present invention may include positioning a first sensor in fluid contact with an analyte of a patient, calibrating the first sensor, positioning a second sensor in fluid contact with the analyte of the patient after calibrating the first sensor, calibrating the second sensor based on data from the first sensor, and removing the first sensor while retaining the second sensor in fluid contact.

The second sensor in one embodiment may be subcutaneously placed in the patient after a predetermined time period has passed where the first sensor has been in fluid contact with an analyte of a patient.

In one embodiment, the stability of the second sensor may be verified by correlation of its output with the output of the stabilized first sensor, prior to calibration of the second sensor based on data from the first sensor, and thereafter removing the first sensor while retaining the second sensor in fluid contact.

A system for determining the stability of an analyte sensor calibration in accordance with still yet another embodiment includes a first sensor for subcutaneous placement in a patient, and a second sensor for subcutaneous placement in the patient after calibration of the first sensor, where at least a portion of the first sensor and at least a portion of the second sensor are in fluid contact with the patient's analyte substantially simultaneously for a time period, and further, where the stability of the second sensor is determined with reference to data from the first sensor.

The time period may be predetermined and includes approximately 2 hours to 10 hours.

Alternatively, the time period may be variable, and where the variable time period may be determined to be when the analyte levels measured by the first and second sensors are within a correlation range which may be determined by a preset threshold value.

A system for determining analyte concentrations in yet another embodiment includes a plurality of analyte sensors, a plurality of transmitter units, each of the plurality of transmitter units operatively coupled to a respective one of the plurality of analyte sensors, a single receiver unit configured to receive and process data substantially simultaneously from all of the plurality of transmitter units, where each transmitter is uniquely couple to a single analyte sensor.

The receiver unit may also include a comparison unit for comparing the one or more signals from the plurality of transmitter units, and also for determining the stability of the plurality of sensors. In addition, the receiver unit may be further configured to determine the calibration of the plurality of sensors based on the comparison unit.

Various other modifications and alterations in the structure and method of operation of this invention will be apparent to those skilled in the art without departing from the scope and spirit of the invention. Although the invention has been described in connection with specific preferred embodiments, it should be understood that the invention as claimed should not be unduly limited to such specific embodiments. It is intended that the following claims define the scope of the present invention and that structures and methods within the scope of these claims and their equivalents be covered thereby.

What is claimed is:

1. A method, comprising:
receiving data from a first sensor and data from a second sensor substantially simultaneously, the first and second sensors both configured to be positioned in fluid contact with bodily fluid under a skin surface, wherein the data from the first sensor and the data from the second sensor correspond to analyte levels in the bodily fluid;
applying a scaling factor to the data from the second sensor to obtain scaled data;
after applying the scaling factor, determining a correlation level between the data from the first sensor and the scaled data;
determining that the correlation level is above a predetermined threshold;
in response to determining that the correlation level between the data from the first sensor and the scaled data is above the predetermined threshold, determining that the second sensor is stable and assigning a calibration associated with the first sensor to the second sensor.

2. The method of claim 1, wherein the calibration includes a calibration code.

3. The method of claim 1, wherein the scaling factor is selected to yield a lowest level of average error between the data from the first sensor and the data from the second sensor.

4. The method of claim 1, wherein the scaling factor is determined by selecting the scaling factor from a group of predetermined scaling factors.

5. The method of claim 1, wherein the scaling factor is determined by performing an autocorrelation function.

6. The method of claim 1, further comprising positioning the second sensor in fluid contact with the bodily fluid after the first sensor is positioned in fluid contact with the bodily fluid.

7. The method of claim 1, further comprising calibrating the second sensor using the data from the first sensor as a reference.

8. The method of claim 7, wherein calibrating the second sensor is performed without a user-initiated reference measurement.

9. The method of claim 1, furthering comprising calibrating the second sensor using the scaling factor and the data from the first sensor.

10. The method of claim 1, wherein the data from the first sensor and the data from the second sensor are received over different kinds of communication links, the kinds of communication links selected from a list including a radio frequency link, a Bluetooth link, an infrared link, or a wired link.

11. The method of claim 1, further comprising determining a sensor sensitivity for the second sensor based on the scaling factor.

12. The method of claim 3, wherein the correlation level comprises a measure for the degree of correspondence between the analyte levels corresponding to the data from the first sensor and the scaled analyte levels corresponding to the data from the second sensor over a period of time.

13. The method of claim 1, wherein the analyte is glucose.

14. The method of claim 1, wherein the analyte is ketone.

15. The method of claim 1, wherein the analyte is lactate.

16. A system, comprising:
a first sensor and a second sensor both configured to be positioned in fluid contact with bodily fluid under a skin surface;
a first sensor electronics operatively coupled to the first sensor;
a second sensor electronics operatively coupled to the second sensor; and
a receiving device, comprising:
one or more processors; and
a memory storing instructions which, when executed by the one or more processors, cause the one or more processors to:
substantially simultaneously receive data from the first sensor and the second sensor, wherein the data from the first sensor and the data from the second sensor correspond to analyte levels in the bodily fluid;
apply a scaling factor to the data from the second sensor to obtain scaled data;
after applying the scaling factor, determine a correlation level between the data from the first sensor and the scaled data;
determine that the correlation level is above a predetermined threshold;
in response to determining that the correlation level between the data from the first sensor and the data from the second sensor is above the predetermined threshold, determine that the second sensor is stable and assign a calibration associated with the first sensor to the second sensor.

17. The system of claim 16, wherein the calibration includes a calibration code.

18. The system of claim 16, wherein the scaling factor is selected to yield a lowest level of error between the data from the first sensor and the data from the second sensor

19. The system of claim 16, wherein the scaling factor is determined by selecting the scaling factor from a group of predetermined scaling factors.

20. The system of claim 16, wherein the scaling factor is determined by performing an autocorrelation function.

21. The system of claim 16, wherein the second sensor is positioned in fluid contact with the bodily fluid after the first sensor is positioned in fluid contact with the bodily fluid.

22. The system of claim 16, the memory storing instructions to calibrate the second sensor using the data from the first sensor as a reference.

23. The system of claim 22, the memory storing instructions to calibrate the second sensor without a user-initiated reference measurement.

24. The system of claim 16, the memory storing instructions to calibrate the second sensor using the scaling factor and the data from the first sensor.

25. The system of claim 16, wherein the data from the first sensor and the data from the second sensor are received over different kinds of communication links, the kinds of communication links selected from a list including a radio frequency link, a Bluetooth link, an infrared link or a wired link.

26. The system of claim 16, the memory storing instructions to determine a sensor sensitivity for the second sensor based on the scaling factor.

27. The system of claim 16, wherein the correlation level comprises a measure for the degree of correspondence between the analyte levels corresponding to the data from the first sensor and the analyte levels corresponding to the data from the second sensor over a period of time.

28. The system of claim 16, wherein the analyte is glucose.

29. The system of claim 16, wherein the analyte is ketone.

30. The system of claim 16, wherein the analyte is lactate.
